(19) United States
(12) Patent Application Publication
(10) Pub. No.: US 2005/0197554 A1
(43) Pub. Date: Sep. 8, 2005

(54) Composite thin-film glucose sensor

(76) Inventor: Michael Polcha, Lovettsville, VA (US)

Correspondence Address: EDELL, SHAPIRO & FINNAN, LLC, 1901 RESEARCH BOULEVARD, SUITE 400, ROCKVILLE, MD, 20850, US

(21) Appl. No.: 11/066,619

(22) Filed: Feb. 28, 2005

Related U.S. Application Data
(60) Provisional application No. 60/547,434, filed on Feb. 26, 2004.

(51) Int. Cl. A61B 5/00

(52) U.S. Cl. 600/365

(57) ABSTRACT

A sensor system including a holder with at least one semi-permeable layer that forms a chamber, at least one reactant that reacts with at least one analyte, the at least one reactant being contained within the chamber, and a detector disposed proximate the at least one semi-permeable layer and configured to detect and measure a concentration of a reaction product from reaction of the at least one reactant with the at least one analyte. The at least one semi-permeable layer allows passage of the analyte into the chamber and allows passage of the reaction product to the detector. In a preferred embodiment, the analyte includes glucose, and the reaction product detected includes carbon dioxide.

### CROSS REFERENCE TO RELATED APPLICATION

This application claims priority from U.S. Provisional Patent Application Ser. No. 60/547,434, entitled “Composite Thin-Film Glucose Sensor”, filed Feb. 26, 2004. The disclosure of this provisional patent application is incorporated herein by reference in its entirety.

### BACKGROUND OF THE INVENTION

1. Field of the Invention

The present invention relates to the detection and concentration of an analyte, in particular glucose, present within a mammal.

2. Description of the Related Art

In many cases the level of the chemical constituents of the body, particular tissues, or the blood are actively controlled. Such control requires that there be a sensor that responds to changes in constituent concentrations and that the sensor relays the constituent information to appropriate cells or tissues that can act to correct the situation. These sensors are usually living cells that are specialized to react to a specific chemical stimulus. In some cases the cell sensors activate a nerve that transmits the constituent information to appropriate tissues that generate the correction. For example, an increase in blood carbon dioxide levels activates sensors that in turn activate a response that eventually results in a change in lung ventilation.

In some cases sensor cells themselves act to correct the constituent levels. For example, alfa and beta cells of the pancreas respond to changes in the constituent levels and then secrete various hormones that affect, among other things, the constituent level.

The blood levels of chemical constituents such as sex-linked hormones (estrogens, androgens, etc.); metabolism-controlling hormones (thyroid, growth hormones, etc.); and steroids, etc., are also detected by cells with special sensitivity to a specific substance or group of substances. The detection of these hormones then results in a corrective response being generated.

Occasionally sensors respond to a change in constituent concentration by relaying information to the nervous system, but such information is not acted upon. For example, the chemo-sensors in the taste buds or the olfactory (smell) systems relay information to the nervous system but no corrective measures necessarily result. Nonetheless, these types of cells are excellent chemo-sensors.

Numerous diseases and pathophysiological states are associated with deviations from normal concentrations of constituents in the blood and bodily tissues. For instance, an elevation of blood and tissue potassium ion and urea levels is associated with many kidney diseases; an elevation of blood glucose levels is associated with diabetes; lowering of thyroxin levels is associated with various thyroid gland malfunctions.

Blood glucose monitoring is crucial in the estimation, calculation, and monitoring of metabolic rate. Metabolic rate monitoring has many clinical applications ranging from therapeutic/diagnostic for obesity and diabetes to caloric requirements for critically ill patients and individuals in training and stressful situations. The rapid and timely use of metabolic data both in the field and in clinical situations will produce dramatic results towards improving quality of life and saving lives.

Careful metabolic monitoring and proper treatment can improve control of diseases such as diabetes and obesity. Knowing a patient's metabolism along with other physiological parameters allows for correct dosing and delivery of medications and nutrients. Improvements in metabolic measurement technology are essential for better diagnostics and advances in treatments of metabolic diseases and conditions. Treatments of metabolic diseases and conditions ideally require frequent and timely monitoring which drive a need for monitors that are non-invasive, real-time, portable, low cost, and accurate. Metabolic data is also useful in assessing the physiological homeostatic conditions of patients and healthy subjects in general.

Blood glucose concentration data is extremely useful for the control of diseases such as diabetes and for monitoring the overall metabolic condition of a human subject. An accurate, real-time, non-invasive method for measurement of blood glucose levels is of the greatest interest in the diabetic communities. Current technologies involving the measurement of blood glucose by probe tend to be invasive. Measurement by probe involves frequent lancing and results in many long-term problems. An ideal non-invasive blood glucose sensor is one that produces an electrical signal that can be used to control devices, such as insulin pumps in closed loop feedback applications. Ongoing development efforts to address the need for non-invasive blood glucose measurements are dependant on breakthroughs in material science and biochemistry. The development of this proposed sensor technology should be free of the impediment of required breakthroughs in advanced research.

There remains, however, a need for improved biosensors. For example, there remains a need for developing new and innovative technology solutions in analyte, e.g., blood glucose, monitoring. Accordingly, there also remains a need for methods of making and using improved biosensors.

### SUMMARY OF THE INVENTION

Therefore, in light of the above, and for other reasons that become apparent when the invention is fully described, an object of the present invention is to provide a sensor for measuring an analyte, such as glucose, in the body of a mammal that is non-invasive and reliable.

It is another object of the present invention to provide such a sensor that is easy and relatively inexpensive to manufacture.

It is a further object of the present invention to provide a system incorporating the sensor that facilitates delivery of a select component (e.g., insulin) to the body of the mammal in response to the measured analyte concentration as determined by the sensor.

The aforesaid objects are achieved individually and in combination, and it is not intended that the present invention be construed as requiring two or more of the objects to be combined unless expressly required by the claims attached hereto.

In accordance with the present invention, a sensor system comprises a holder including at least one semi-permeable layer that forms a chamber, at least one reactant that reacts with at least one analyte, the at least one reactant being contained within the chamber, and a detector disposed proximate the at least one semi-permeable layer and configured to detect and measure a concentration of a reaction product from reaction of the at least one reactant with the at least one analyte. The at least one semi-permeable layer allows passage of the analyte into the chamber and allows passage of the reaction product to the detector.

In a preferred embodiment, the analyte includes glucose, and the reaction product detected includes carbon dioxide.

The above and still further objects, features and advantages of the present invention will become apparent upon consideration of the following detailed description of specific embodiments thereof.

### BRIEF DESCRIPTION OF THE DRAWINGS

FIG. 1 is an exploded view of a composite thin-film glucose sensor in accordance with the present invention.

### DESCRIPTION OF THE PREFERRED EMBODIMENTS

Unless otherwise stated, all references to a compound or component include the compound or component by itself, as well as in combination with other compounds or components, such as mixtures of compounds. As used herein, the singular forms “a,” “an,” and “the” include the plural reference unless the context clearly dictates otherwise. In addition, the term “reactant”, as used herein, refers to any substance or material that take part in a chemical reaction to yield a detectable component that correlates with the concentration of an analyte of interest (e.g., glucose). The reactant can include, for example, a chemical compound, including catalysts such as enzymes. Alternatively, or in combination with chemical compounds, the reactant can include living organisms or cells, such as micro-organisms, bacteria, yeast, etc. The preferred reactant of interest, discussed in further detail below, is yeast (e.g., baker's or brewer's yeast).

The sensor of the present invention includes at least one reactant that reacts with at least one analyte. In general, the sensors of the present invention are useful in conjunction with any reactive system that may be contained within a chamber of a holder for a determination of the presence and/or concentration of an analyte in a test sample. Thus, the reactant may be used to monitor the presence and/or concentration of a broad range of analytes. Examples of reactants include those that are sensitive to analytes such as, but are not limited to, carbohydrates (e.g., glucose, glycogen, fructose, mannose, sucrose), lipids (e.g., cholesterol, lipid acids, high and low density lipids), creatinine, lactate, enzymes (e.g., ATP, dehydrogenases, lipases, trypsin), amino acids, peptides and proteins (albumins, polypeptides, antibodies, antigens), electrolytes (e.g., ions of sodium, potassium, calcium, hydrogen, chloride), coumarin, hormones (e.g., thyroid, steroids, insulin, glucagon, adrenaline, synthroid, erythropoietin), cytokines (e.g., chemokines), toxins (e.g., endotoxins, pertussis toxin, tetanus, toxin), transmitters (e.g., acetyl choline, GABA), volatile substances that are recognized by smell (e.g., alcohols, ethers, esters), water dissolved substances that are recognized by taste (e.g., sugars, carbohydrates, amino acids), dissolved gases (e.g., O₂, CO₂, nitrogen, carbon monoxide, hydrogen), antibiotics (e.g., cyclosporin), and other drugs (e.g., lopid, monopril, digoxin, amiodarone, prothrombin, various chemotherapeutic drugs, such as taxol and fluorouracil). In a particularly preferred embodiment, the at least one reactant may be one that reacts with glucose. Accordingly, the present invention may be used to monitor a broad range of analytes.

Examples of the at least one reactant include chemical compounds (e.g., enzymes) and living organisms or cells. In several preferred embodiments, the at least one reactant includes at least one living organism or cell. For example, the sensor can include living cells that are sensitive to the concentration of an analyte and that produce signals proportional to concentration changes. Living matter such as animal, plant, bacteria, or fungi cells, or parts thereof may be used to metabolize the analyte, where an metabolized output (e.g., chemical compounds produced by biochemical reactions) by the living matter can be detected and correlated with analyte concentration. For example, the at least one organism can be one that reacts with oxygen, water, and glucose. Further, the at least one organism can produce carbon dioxide, which is detected and correlated with analyte concentration.

In a preferred embodiment, the at least one organism includes yeast. A variety of yeasts (e.g., normal or abnormal if in a disease state) are found living on human skin. Preferably, the yeast should be a variety that has a tendency to be robust and stable in a skin surface sensor configuration. Yeasts such as this grow rapidly and thrive on glucose as a food source and obtain suitable amounts of moisture and oxygen as well from the skin. The yeast naturally produces carbon dioxide as a by-product.

Strains of yeast cells are known which metabolize glucose. In fact, some strains of yeast cells are known which metabolize only glucose and not other substances, thus enabling a sensor using such yeast cells to be highly selective. Such strains of yeast are disclosed, e.g., in “The Yeasts” edited by Jacomina Lodder, published by North-Holland Publishers, Amsterdam, 1970, the disclosure of which is incorporated herein by reference in its entirety. The yeast can be, e.g., a baker's or brewer's yeast. Because the yeast cells stay alive even after being included in the sensor, the yeast cells are self-renewing, thereby allowing the sensor to have an extended lifetime.

In another embodiment, the at least one organism includes at least one luminescent organism. For instance, the at least one luminescent organism can be a genetically modified or recombinant yeast that luminesces (e.g., a yeast genetically modified with firefly luciferase).

In some embodiments, the at least one reactant includes chemical sensor cells in taste buds that respond to fluctuations in glucose, salts, and other analytes. See, e.g., OZEKI, J. Gen. Physiol., 58:688-699 (1971); AVENET et al., J. Membrane Biol., 97:223-240 (1987); and TONOSAKI et al.; Brain Research, 445:363-366 (1988), the disclosures of which are incorporated herein by reference in their entireties. Under suitable conditions, taste cells regenerate every few days by continuous division. Thus, prolonged growth of these cells within the sensor of the present invention is more readily sustained. Taste cells are also more accessible than other cells. A sample of taste cells can be removed from a patient with only minor surgery, grown in culture to obtain a sufficient number of cells, and then inserted into the sensor. The ability to use a patient's own cells also reduces the possibility of an immune reaction in case the cells escape the sensor.

In certain embodiments, the at least one reactant includes Alpha cells from the pancreas that are sensitive to glucose as well as other analytes. See, e.g., SONERSON et al., Diabetes, 32:561-567 (1983), the disclosure of which is incorporated herein by reference in its entirety. Transformed cell lines, such as the insulin producing line disclosed in U.S. Pat. No. 4,332,893, which is incorporated herein by reference in its entirety, and hybridoma lines can also be used. In preferred embodiments, electrical activity associated with the response by Alpha cells or transformed lines can be harnessed in practicing the present invention.

In certain embodiments, Beta cells from the islets of Langerhans in the pancreas are used as glucose sensitive cells. Beta cells have been shown to produce electrical activity, action potentials, in response to glucose concentration and have the advantage that they respond properly to glucose in the concentration range relevant to patient monitoring. See, e.g., SCOTT et al., Diabetologia, 21.470-475(1981); PRESSEL et al., Biophys. J, 55:540a (1989); and HIDALGO et al., Biophys. J, 55:436a (1989); ATWATER et al., Biophys. J, 55: 7a (1980), the disclosures of which are incorporated herein by reference in their entireties. Beta cells respond to glucose in bursts of spikes of electrical activity. The spike frequency, burst duration and pauses between bursts are all functions of glucose concentration. The burst duration increases as glucose concentration increases. The pause between bursts also decreases as glucose concentration increases. The spike frequency (spikes/second) increases as glucose concentration increases. Each of these parameters (burst duration, pause duration and spike frequency), as well as spike shape, can be monitored alone or in combination as a source of signal corresponding to cellular electrical activity. It has also been established that the beta cells are electrically coupled, resulting in synchronized electrical activity of the cells. EDDIESTONE et al., J. Membrane Biol., 77:1-141 (1984), MEDA et al., Quarterly J. Exper. Physiol., 69:719-735 (1984), the disclosure of which is incorporated herein by reference in its entirety. Therefore, in response to a change in the glucose concentration, many cells fire their action potentials or electrical signals in synchrony, producing a significantly amplified signal that is easier to detect.

In embodiments where the at least one reactant includes at least one organism, the response of the sensor depends on how quickly the at least one organism reacts to its environment. The reproductive activity of the organism will also be a factor. Yeasts, which tend to grow quickly and rapidly respond to their environmental conditions, are preferred. In preferred embodiments, the at least one organism is typically held in a controlled and constrained space, so its growth will be limited. In these cases, the geometry of the constrained volume will determine sensor sensitivity and full-scale saturation levels. In some embodiments, the metabolic results will increase and decrease directly due to waxing and waning of organism populations, which will be driven by the concentration of metabolic inputs and organism reproduction time.

In certain embodiments, the at least one reactant includes one or more enzymes. Enzymes are biological catalysts and many of them have an unusual specificity for catalyzing a particular reaction with a single, specific and predetermined chemical substance. Examples of suitable enzymes include, but are not limited to, oxidase enzymes such as glucose oxidase, cholesterol oxidase, uricase, alcohol oxidase, aldehyde oxidase, and glycerophosphate oxidase.

In a preferred embodiment, the analyte reacts with a specific oxidase enzyme to produce hydrogen peroxide. This strongly oxidative substance reacts with indicator(s) present to produce a colored end product.

For instance, the present invention contemplates glucose measurements using a glucose enzyme and a substance capable of undergoing a color change with one or more of the compounds formed during the reaction of the enzyme with glucose. The compounds formed during the reaction involving glucose may in turn react with other substances which themselves undergo no color change or only a slight color change but which react with a color-forming substance to produce a color. More than one substance can mediate between the compounds formed during the reaction and the color-forming substance. Preferred glucose enzymes include those that catalyze a reaction of glucose to produce a predetermined reaction product. The indicating substance is one capable of forming a color or changing color in the presence of a reaction product or a mediating substance.

In a preferred embodiment, a color-forming substance is incorporated into the reactant system which will be oxidized or reduced by any hydrogen peroxide formed, or reduced by reduced flavin present in glucose oxidase, in the fluid medium as a result of reaction between glucose, glucose oxidase, and oxygen to produce a colored material or a material of a different color from that of the original substance. The color-forming substance can undergo color change not as a result of direct action of the hydrogen peroxide but can be mediated through another compound which is acted upon by the hydrogen peroxide but which does not itself become highly colored.

In accordance with a preferred embodiment of the invention, the reactant system contains a dual enzyme system, one enzyme of which catalyzes the transformation of glucose to produce hydrogen peroxide, the other enzyme having peroxidase activity, where the indicator also includes a color-forming substance which is sensitized when hydrogen peroxide is produced as a result of glucose being present.

Suitable antibody assay labels are known in the art and include enzyme labels, such as glucose oxidase; luminescent labels, such as luminol; and fluorescent labels, such as fluoroscein, rhodamine, and biotin. For instance, the analyte may be monitored by using the reaction system disclosed in U.S. Pat. No. 6,454,710, which is incorporated herein by reference in its entirety.

The at least one reactant of the present invention can be contained in a reaction medium. Substantially any reaction medium may be used so long as it does not interfere with the reaction of interest. Examples of reaction media include, but are not limited to, water, aqueous solutions, and gels. Methods for immobilizing yeast in a solid gel are known. See, e.g., KUU et al., “Improving Immobilized Biocatalysts by Gel Phase Polymerization” Biotechnology and Bioengineering, Vol. XXV, 1995-2006 (1983), the disclosure of which is incorporated herein by reference in its entirety.

The amount of the at least one reactant in the sensor of the present invention is not particularly limited, so long as there is sufficient reactant to cause a degree of reaction sufficient to produce a detectable change. For example, the amount of at least one reactant can range from about 1000 units to about 100,000 units per 20 grams of reaction system material. When the at least one reactant includes cells, the reaction system preferably includes from about 2,000 to about 50,000 individual cells, more preferably about 7,500 to about 12,500 cells. When color forming agents are present, the amount of color forming or color changing agents may, e.g., range from about 0.01 wt % to 30 wt %, based on the total weight of reaction system. While the pH of the reaction system is not particularly limited, the pH of the reaction system may, e.g., range from about 7 to about 11, or about 8 to about 10.

The at least one reactant is typically contained within a chamber of a holder for the sensor. The chamber can be formed by at least one semi-permeable layer. In a preferred embodiment, the at least one semi-permeable membrane surrounds the chamber. In an exemplary embodiment, the at least one semi-permeable membrane includes at least two semi-permeable membranes that are bonded together to form the chamber.

The at least one semi-permeable layer allows passage of the analyte (e.g., from the skin of the user) into the chamber and allows passage of the reaction product or products from the layer (e.g., to a detector of the sensor). Thus, the at least one semi-permeable membrane serves as a barrier that prevents the at least one reactant from migrating away, while nutrients and waste products are free to diffuse through the at least one semi-permeable membrane. The at least one semi-permeable membrane also serves to prevent antibodies and other large molecules from leaving or entering the chamber, for example, to prevent immune reactions from occurring within the chamber.

The at least one semi-permeable membrane can include pores for enabling nutrients and waste materials to diffuse to and from the at least one reactant. In preferred embodiments, the semi-permeable membrane is permeable to relatively small molecules, such as up to molecular weights ranging from about 30,000 to about 50,000, and impermeable to larger molecules, such as proteins and antibodies. The porosity of the semi-permeable membrane may be the minimum necessary for the maintenance of the at least one reactant. In other words, in certain embodiments, the semi-permeable membrane permits the inward diffusion of nutrients and O₂, and the outward diffusion of metabolites and CO₂ and excretions, that are sufficient to support long term survival of living organisms or cells that constitute the at least one reactant. Further, the semi-permeable membrane preferably has high biocompatibility with the mammal and the living organisms or cells with which it is associated.

In a preferred embodiment, the at least one semi-permeable membrane of the holder is permeable to glucose but impermeable to body cells, sensor cells, proteins, etc. In other preferred embodiments, the at least one semi-permeable membrane is permeable to oxygen, water, and glucose.

In some embodiments, the at least one semi-permeable membrane allows the use of chemo-sensitive tumor cell lines as the at least one reactant, while the tumor cell lines are contained to prevent proliferation.

Any material that will provide the above-described functions is suitable for use as a semi-permeable membrane for the sensor device of the present invention. Examples of semi-permeable membrane materials for use in constructing the sensor device of the present invention include, without limitation, cellulose acetate, silicones, fluorosiloxanes, polysulfones, polycarbonates, poly(vinyl chlorides) (e.g., PVC/PAN (polyvinylchloride/polyacrylonitrile) polymers such as a polyvinyl chloride acrylic copolymer), polyamides, ethylene vinyl acetate copolymers, poly(vinylidene) fluoride, poly(urethanes), poly(benzimidazoles), cellulose esters, cellulose triacetate, cellulose, cellulose nitrate, regenerated cellulose, cross-linked poly(vinylpyrrolidone); crosslinked polyacrylamide, crosslinked poly(hydroxy ethyl methacrylate), silicones, fluorosiloxanes, PTFE, and combinations thereof. In a preferred embodiment, the semi-permeable membrane includes cellulose acetate.

As noted above, the at least one semi-permeable membrane can include one or more layers. When more than one layer is present, the materials can be the same or different. For example, one material may be coated with a biocompatibility-promoting substance, such as polyethylene glycol, basic fibroblast growth factor, or an angiogenic substance. As another example, the semi-permeable membrane can include a permeable-structural layer and a discriminating semi-permeable portion having a thickness ranging, e.g., from about 1 micron to about 2 microns.

The thickness of the semi-permeable membrane is not particularly limited. For example, the semi-permeable membrane can have a thickness ranging from about 10 microns to about 200 microns, about 15 to about 100 microns. Preferably, the thickness is about 20 microns.

The size of the holder is also not particularly limited. The holder can have a diameter of ranging from about 0.05 mm to about 1.0 mm, preferably from about 0.1 mm to 0.4 mm, such as in the range of about 0.2 mm.

The sensor of the present invention includes a detector that detects reaction product from reaction of the at least one reactant (e.g., with the at least one analyte), and the reaction product correlates with a concentration of analyte. The detectors of the present invention are not particularly limited; i.e., the detector system will depend on the particular reaction system. For example, in certain embodiments, the detector includes a carbon dioxide detector. A carbon dioxide detector is particularly useful in sensor embodiments in which the analyte for detection is glucose. For example, the sensor can be designed such that the rate of change in detected carbon dioxide correlates with changes in the amount of glucose entering the chamber of the sensor, which in turn is correlated with the level of glucose in the mammal's bloodstream to which the sensor device is associated.

Carbon dioxide detectors can be constructed using several known techniques. In one embodiment, the detector measures carbon dioxide concentrations by measuring changes in pH in an aqueous layer. In this regard, carbon dioxide dissolves in water and reversibly forms carbonic acid, which cause a measurable shift in pH levels. In another embodiment, a carbon dioxide-responsive electrode provides an output signal. The detector can be powered by any suitable power source (e.g., battery or other power source to which the detector is connected).

In other embodiments, the detector includes a light detector. Specifically, the detector detects light produced by the reaction. In preferred embodiments, the detector converts the light into an electrical signal. The light detector is used in embodiments in which the reactant (e.g., organisms or cells) luminesces in response to the presence of particular analytes. For instance, gene-splicing technology could be used to produce a variety of skin compatible yeasts that would have luminescent properties. As noted above, yeast cells can be genetically modified to include firefly luciferase. The quantity of the light emitted by the genetically modified yeast could be used as a measure of glucose levels. For example, the amount of luminescence by the genetically modified yeast cells correlates to the metabolic activity of the yeast cells, which in turn correlates with the amount of glucose with which the yeast cells react. Thin film detectors, which are sensitive to light, could be easily constructed to directly produce electrical signals proportional to detected light levels. In certain embodiments, the detector monitors electrical signals from the reaction. Such detectors are known in the art, e.g., as disclosed in U.S. Pat. No. 6,091,974, the disclosure of which is incorporated herein by reference in its entirety.

In certain embodiments in which heat is generated as a result of reactions taking place in the chamber (e.g., between reactant and analyte), the detector monitors heat generated by the reaction. Such detectors are known in the art, e.g., as disclosed in U.S. Pat. No. 4,935,345, the disclosure of which is incorporated herein by reference in its entirety. In this regard, the heat of reaction creates a temperature differential that is detected by sensing and reference junctions of a microelectronic biochemical sensor in order to provide an indication of the concentration of the analyte within the chamber of the sensor. For instance, the heat of metabolism associated with the reduction of glucose by yeast may be greater than the corresponding heat of reaction associated with the chemical reduction of glucose by enzymes, because yeast is capable of decomposing glucose completely to ethanol.

Thus, in preferred embodiments, the detector is able to quantify the reaction of the at least one organism with the at least one analyte and determine analyte level.

In certain transdermal applications, the sensors of the present invention are provided with a mechanism for holding the sensor close to the skin or mucosa of a mammal. For instance, the semi-permeable layer can itself be an adhesive. The sensor can also include an adhesive layer that holds the sensor to a skin or mucosa surface of a mammal.

In preferred embodiments, the adhesive adheres instantaneously to most surfaces with the application of very slight pressure and remains permanently tacky. Examples of suitable adhesives include, without limitation, all of the non-toxic natural and synthetic polymers known for or suitable for use in transdermal devices as adhesives including acrylic polymers, gums, silicone-based polymers (broadly referred to as “polysiloxanes”) and rubber-based adhesives such as polyisobutylenes, polybutylenes, ethylene/vinyl acetate and vinyl acetate based adhesives, styrene/butadiene adhesives, polyisoprenes, styrenes and styrene block copolymers and block amide copolymers. Suitable polysiloxanes include, without limitation, silicone pressure-sensitive adhesives that are based on two major components: a polymer, or gum, and a tackifying resin. The polysiloxane adhesive can be prepared by cross-linking the gum, preferably a high molecular weight polydiorganosiloxane, with the resin, to produce a three-dimensional silicate structure, via a condensation reaction in an appropriate organic solvent. The ratio of resin to polymer is the most important factor that can be adjusted in order to modify the physical properties of polysiloxane adhesives. See, e.g., SOBIESKI et al., “Silicone Pressure Sensitive Adhesives,” Handbook of Pressure-Sensitive Adhesive Technology, 2nd ed., pp. 508-517 (D. Satas, ed.), Van Nostrand Reinhold, New York (1989), the disclosure of which is incorporated herein by reference in its entirety.

Further details and examples of silicone pressure-sensitive adhesives that are useful in the practice of the present invention are described in U.S. Pat. Nos. 4,591,622, 4,584,355, 4,585,836 and 4,655,767, the disclosures of which are incorporated herein by reference in their entireties. Examples of suitable silicone pressure-sensitive adhesives that are commercially available include the silicone adhesives sold under the trademarks BIO-PSA® by Dow Corning Corporation (Midland, Mich.).

In particularly preferred embodiments of the invention, the adhesive matrix composition comprises a pressure-sensitive adhesive, and more preferably a blend of one or more pressure-sensitive acrylic polymers and polysiloxanes. Acrylic polymers include, without limitation, acrylate polymer, polyacrylate, and polyacrylic adhesive polymers as used herein and as known in the art. The acrylic polymers further include polymers of one or more monomers of acrylic acids and other copolymerizable monomers. The acrylic polymers also include copolymers of alkyl acrylates and/or methacrylates and/or copolymerizable secondary monomers or monomers with functional groups. By varying the amount of each type of monomer added, the cohesive properties of the resulting acrylic polymer can be changed as is known in the art. It is preferred to provide an acrylic polymer that is composed of at least 50% by weight of an acrylate or alkyl acrylate monomer, from 0 to 20% of a functional monomer copolymerizable with the acrylate, and from 0 to 40% of other monomers.

Acrylate monomers that can be used include acrylic acid, methacrylic acid, butyl acrylate, butyl methacrylate, hexyl acrylate, hexyl methacrylate, 2-ethylbutyl acrylate, 2-ethylbutyl acrylate, 2-ethylbutyl methacrylate, isooctyl acrylate, isooctyl methacrylate, 2-ethylhexyl acrylate, 2-ethylhexyl methacrylate, decyl acrylate, decyl methacrylate, dodecyl acrylate, dodecyl methacrylate, tridecyl acrylate, and tridecyl methacrylate.

Functional monomers, copolymerizable with the above alkyl acrylates or methacrylates, which can be used include acrylic acid, methacrylic acid, maleic acid, maleic anhydride, hydroxyethyl acrylate, hydroxypropyl acrylate, acrylamide, dimethylacrylamide, acrylonitrile, dimethylaminoethyl acrylate, dimethylaminoethyl methacrylate, tert-butylaminoethyl acrylate, tert-butylaminoethyl methacrylate, methoxyethyl acrylate and methoxyethyl methacrylate and other monomers having at least one unsaturated double bond which participates in copolymerization reaction in one molecule and a functional group on its side chain such as a carboxyl group, a hydroxyl group, a sulfoxyl group, an amino group, an amino group and an alkoxyl, as well as a variety of other monomeric units including alkylene, hydroxy-substituted alkylene, carboxylic acid-substituted alkylene, vynylalkanoate, vinylpyrrolidone, vinylpyridine, vinylpirazine, vinylpyrrole, vinylimidazole, vinylcaprolactam, vinyloxazole, vinylacate, vinylpropionate and vinylmorpholine.

Further examples of acrylic adhesives that are suitable in the practice of the invention are described in SATAS, “Acrylic Adhesives,” Handbook of Pressure-Sensitive Adhesive Technology, 2.sup.nd ed., pp. 396-456 (D. Satas, ed.), Van Nostrand Reinhold, New York (1989), the disclosure of which is incorporated herein by reference in its entirety.

Suitable acrylic adhesives are commercially available and include the polyacrylate adhesives sold under the trademarks DURO-TAK® by National Starch Company (Bridgewater, N.J.), GELVA® by Solutia (St. Louis, Mo.), HRJ by Schenectady International, Inc. (Chicago, Ill.) and EUDRAGIT® by Roehm Pharma GmbH (Darmstadt, Germany).

The adhesive can also contain one or more solvents and/or co-solvents. Such solvents and/or co-solvents are those known in the art, and are non-toxic, pharmaceutically acceptable substances, preferably liquids, which do not substantially negatively affect the adhesive properties or the solubility of the reactants and other active agents at the concentrations used. The solvent and/or co-solvent can be for the active agent or for the adhesive, or both.

Suitable solvents include volatile liquids such as alcohols (e.g., methyl, ethyl, isopropyl alcohols and methylene chloride); ketones (e.g., acetone); aromatic hydrocarbons such as benzene derivatives (e.g., xylenes and toluenes); lower molecular weight alkanes and cycloalkanes (e.g., hexanes, heptanes and cyclohexanes); and alkanoic acid esters (e.g., ethyl acetate, n-propyl acetate, isobutyl acetate, n-butyl acetate isobutyl isobutyrate, hexyl acetate, 2-ethylhexyl acetate or butyl acetate); and combinations and mixtures thereof.

Suitable co-solvents include polyhydric alcohols, which include glycols, triols and polyols such as ethylene glycol, diethylene glycol, propylene glycol, dipropylene glycol, trimethylene glycol, butylene glycol, polyethylene glycol, hexylene glycol, polyoxethylene, glycerin, trimethylpropane, sorbitol, polyvinylpyrrolidone, and the like.

Further suitable co-solvents include glycol ethers such as ethylene glycol monoethyl ether, glycol esters, glycol ether esters such as ethylene glycol monoethyl ether acetate and ethylene glycol diacetate; saturated and unsaturated fatty acids, mineral oil, silicone fluid, lecithin, retinol derivatives and the like, and ethers, esters and alcohols of fatty acids.

Although the exact amount of co-solvents that may be used in the adhesive composition depends on the nature and amount of the other ingredients, such amount typically ranges from about 0.1 wt % to about 40 wt %, such as about 0.1 wt % to about 30 wt %, or about 1 wt % to about 20 wt %, based on the dry weight of the adhesive composition.

In one embodiment, the sensor of the present invention is designed to fit around a body part. In some embodiments, the sensor is adapted to fit on a body location where sweat is generated. In certain embodiments, a thick insulating band with a Velcro fastener may be used to fasten the band to the body. For example, glucose is known to permeate the skin of a mammal via sweat, and such glucose can be correlated with glucose concentration in the blood of the mammal. For example, a method for correlating glucose concentration in blood plasma of a human based upon the concentration of glucose in perspiration or sweat is described in U.S. Pat. No. 5,140,985, the disclosure of which is incorporated herein by reference in its entirety.

In some transdermal sensor embodiments, the sensor may further include a skin or mucosa permeation enhancer adjacent to the holder. Permeation enhancers increase the permeability of skin to interstitial fluid and/or the analyte(s) of interest. For instance, the skin permeation enhancer can be a glucose permeation enhancer.

The permeation enhancer can be a chemical permeation enhancer, a mechanical permeation enhancer, or one or more combinations thereof. For instance, the permeation enhancer can include a chemical skin permeation enhancer or a mixture of chemical skin permeation enhancers; ultrasound; iontophoresis; tape stripping; microtines; electroporation; or a combination thereof.

In general, two or more chemical skin permeation enhancers can be used in combination with each other. Examples of the skin permeation enhancers include, without limitation, natural bile salt, sodium cholate, sodium dodecyl sulfate, sodium deoxycholate, taurodeoxycholate, and sodium glycocholate. Skin permeation enhancers also can include C₂-C₄ alcohols such as ethanol and isopropanol, polyethylene glycol monolaurate, polyethylene glycol-3-lauramide, dimethyl lauramide, esters of fatty acids having from about 10 to about 20 carbon atoms, and monoglycerides or mixtures of monoglycerides of fatty acids having a total monoesters content of at least 51% where the monoesters are those with from 10-20 carbon atoms.

Skin permeation enhancers also include diglycerides and triglycerides of fatty acids, or mixtures thereof. Fatty acids include, for example, lauric acid, myristic acid, stearic acid, oleic acid, linoleic acid, and palmitic acid. Monoglyceride permeation enhancers include glycerol monooleate, glycerol monolaurate, and glycerol monolinoleate, for example. In a preferred embodiment, the permeation enhancer is a polyethylene glycol-3-lauramide (PEG-3LR), glycerol monooleate (GMO), glycerol monolinoleate, or glycerol monolaurate (GML), more preferably, glycerol monooleate. Other preferred permeation enhancers include, but are not limited to, diethylene glycol monoethyl ether, dodecyl acetate, propylene glycol, methyl laurate, ethyl acetate, isopropyl myristate, ethyl palmitate, isopropyl palmitate, glycerol monocaprylate, isopropyl oleate, ethyl oleate, lauryl pidolate, lauryl lactate, propylene glycol monolaurate, n-decyl methyl sulfide. Still other permeation enhancers include vegetable, animal, and fish fats and oils such as cottonseed, corn, safflower, olive and castor oils, squalene, and lanolin.

Permeation enhancers also include polar solvents such as dimethyldecylphosphoxide, methyloctylsulfoxide, dimethyllaurylamide, dodecylpyrrolidone, isosorbitol, dimethylacetonide, dimethylsulfoxide, decylmethylsulfoxide, and dimethylformamide, which affect keratin permeability; salicylic acid which softens the keratin; amino acids which are penetration assistants; benzyl nicotinate which is a hair follicle opener; and higher molecular weight aliphatic surfactants such as lauryl sulfate salts which change the surface state of the skin and drugs administered and esters of sorbitol and sorbitol anhydride such as polysorbate 20 commercially available under the trademark Tween® 20 from ICI Americas, Inc. (Wilmington, Del.), as well as other polysorbates such as 21, 40, 60, 61, 65, 80, 81, and 85. Other suitable enhancers include oleic and linoleic acids, triacetin, ascorbic acid, panthenol, butylated hydroxytoluene, tocopherol, tocopherol acetate, and tocopherol linoleate.

In certain preferred embodiments, the skin permeation enhancers are osmotic agents (e.g., NaCl) to greatly improve the kinetics of interstitial fluid flow. In some cases, osmotic agents improve the flow rate of interstitial fluid over the flow rates obtained through the use of other skin permeation enhancing means such as chemical adjuvants, electrical potential, ultrasound, mechanical penetration, etc.

In certain embodiments of the invention, a permeation enhancer is incorporated into the adhesive composition. If permeation enhancers are incorporated into the adhesive composition, the amount typically ranges up to about 30 wt %, such as from about 0.1 wt % to about 15 wt %, based on the dry weight of the adhesive composition.

In some embodiments, the skin permeation enhancer includes a skin interface layer having tiny tines to compromise the skin so that body fluid can be extracted through the skin.

In addition to permeation enhancers, there can also be incorporated various pharmaceutically acceptable additives and excipients known to those skilled in the art. Such additives include tackifying agents such as aliphatic hydrocarbons, mixed aliphatic and aromatic hydrocarbons, aromatic hydrocarbons, substituted aromatic hydrocarbons, hydrogenated esters, polyterpenes, silicone fluid, mineral oil, and hydrogenated wood rosins. Additional additives include binders such as lecithin which “bind” the other ingredients, or rheological agents (thickeners) containing silicone such as fumed silica, reagent grade sand, precipitated silica, amorphous silica, colloidal silicon dioxide, fused silica, silica gel, quartz and particulate siliceous materials commercially available as Syloid®, Cabosil®, Aerosil®, and Whitelite®, for purposes of enhancing the uniform consistency or continuous phase of the final composition. Other additives and excipients include diluents, stabilizers, fillers, clays, buffering agents, biocides, humectants, anti-irritants, antioxidants, preservatives, plasticizing agents, cross-linking agents, flavoring agents, colorants, pigments, and the like. Such substances can be present in any amount sufficient to impart the desired properties to the carrier composition. Such additives or excipients are typically used in amounts up to 25 wt %, and preferably from about 0.1 wt % to about 10 wt %, based on the dry weight of the adhesive composition.

In a preferred embodiment, a thermal perforation system is incorporated into the sensor to ablate a microscopic portion of the stratum corneum, the topmost layer of skin, so that the interstitium can be exposed. The thermal perforation system can include a micro-heater in close proximity to the skin surface, together with electrical components that control current to the micro-heaters.

The thermal ablation micro-heater can be pulsed with a suitable alternating or direct current to provide local ablation. Control of the duration and intensity of the heating pulse is preferably carried out to achieve ablation of the correct area and depth of a skin surface. The micro-ablation preferably occurs in a confined volume of the stratum corneum of approximately 50 μm×50 μm×30 μm.

In another preferred embodiment of the present invention, minimally invasive transdermal detection is achieved by laser ablation of the stratum corneum layer.

The sensor of the present invention can include miscellaneous layers. For example, the sensor can include a transparent insulator between the holder and the detector. The purpose of this layer is to protect the detector. This layer can be made of materials known in the art.

Further, in embodiments involving an adhesive, the sensor of the present invention can include a release liner. Release liners are known in the art, such as those disclosed in U.S. Pat. Nos. 5,474,787 and 5,656,286, the disclosures of which are incorporated herein by reference in their entireties.

In addition to detecting the presence of an analyte, the sensor of the present invention can be incorporated into a biomedical monitoring system to provide agent or counteragent delivery (e.g., drug delivery, such as insulin) and including feedback control in bursts to maintain concentrations of a specific agent within the body at specific levels throughout the day (e.g., levels that vary on a day-to-day basis and during the day). In other words, the present invention may be used to improve sensor controlled delivery systems by providing the capability to automatically deliver either an agent or counteragent based on continuous sensor readings to maintain the level of a constituent or condition. Examples of such agents or counteragents include, but are not limited to, hormones, heart medication, glucose, and insulin.

In a preferred embodiment, a glucose monitoring system of the present invention is used to directly regulate blood glucose levels by measuring glucose levels via a glucose sensor and controlling the blood glucose levels via delivery of insulin and/or other suitable drugs. Using the underlying principles of this technology and modifying the sensor to allow for control over the metabolic action of the organism, glucose consumption for the purpose of blood glucose regulation can be achieved for subjects who are unable to regulate their levels normally.

Preferably, the glucose monitoring system includes at least one pump (e.g., an electrically controlled pump) connected to the sensor and at least one reservoir connected to the pump. The at least one reservoir can contain, e.g., at least one of insulin, glucose, and glucagons. The system is configured to deliver a controlled volume or a controlled rate of the agent or counteragent into the appropriate body fluid, cavity or tissue, i.e., blood, peritoneal cavity, subcutaneous tissue, etc. The pump can be of any known type, including a piston or piston equivalent (fluid or gas) driven pump, a peristaltic pump, centrifugal pump, etc. In the alternative, the drug delivery in the system can be carried out by controlled diffusion, by an electric current that carries a charged agent, by charged molecules or particles, by magnetic particles, etc.

Methods for obtaining the reactants (e.g., yeast cells) of the present invention are known in the art. For example, methods for isolating cells are described in AMSTERDAM et al, J. Cell Biol., 63:1037-1056 (1979), RICORDI et al., Diabetes, 35:649-653 (1986), and CARRINGTON et al., J. Endocr., 109:193-200 (1986), the disclosures of which are incorporated herein by reference in their entireties. In addition, any other method for isolating cells can be used which preserves the ability of the isolated cells to respond to changes in chemical concentrations. For instance, methods for culturing pancreatic cells are disclosed in AMSTERDAM et al., J. Cell Biol., 63:1037-1073 (1974); AMSTERDAM et al., Proc. Natl. Acad. Sci. USA, 69:3028-3032 (1972), Ciba Foundation Symposium on the Exocrine Pancreas, Reuck and Cameron, ed., p. 23-49 (J. and A. Churchill Ltd., London 1962), and HOWARD et al., J. Cell. Biol., 35:675-684 (1967), the disclosures of which are incorporated herein by reference in their entireties.

Any suitable method or methods of assembling the sensors and systems according to the present invention will be apparent to one skilled in the art, where conventional laminating techniques for application of adhesive to the various layers, heat bonding various layers and similar techniques for assembly of the devices can be used to assemble the various layers and components.

In addition, the present invention further encompasses systems including multiple sensors that are formed on the same supporting substrate to simultaneously sense the presence of a plurality of different chemicals. For instance, in some embodiments, the sensor of the present invention includes a plurality of chambers for separately containing different reactants. In other embodiments, the present invention includes a plurality of reactants in a single chamber.

The sensor device and system of the present invention can be used for a variety of applications. For example, in certain embodiments, the system can be adapted for monitoring a single analyte or simultaneous monitoring of multiple analytes by including reactants matched to each of the analytes of interest.

In an exemplary embodiment, the sensor of the present invention monitors the health of an individual. For example, the present invention can be used for monitoring a subject for pesticide exposure, monitoring the stress status of a soldier; phenotyping by using the enzyme N-acetyl transferase to indicate an infected or diseased state, monitoring external exposure and internal contamination of a person with either organophosphate nerve agents (tabun, sarin, soman) or organophosphate insecticides (parathion and metabolites thereof), monitoring inflammatory sequeli in response to microbial infection (interleukin-1, interleukin-6, tumor necrosis factor), monitoring microbial toxins (anthrax, botulinum, endotoxin), monitoring spore metabolites arising from human catabolism via lymphatic or hepatic pathways, monitoring stimulants such as caffeine, antihistamines (dexornethorphan, caffeine), and monitoring stress through alterations in blood glucose concentration or altered metabolism of insulin/glucose.

In some embodiments, the sensor of the present invention can be configured as an implant for a mammal for use transdermally. In this case, the sensor can be implanted in the bloodstream or in tissues in equilibrium with blood concentration levels.

The sensors of the present invention can also be used to measure analytes in other body fluids such as sweat. To perform measurements of this type, the sensor is placed in tight contact with the skin. One form for a sensor operative on sweat would be a wristwatch type sensor.

In a preferred embodiment, as discussed above, the present invention further includes a system for delivering drugs in response to the aforementioned assessment of a subject's medical condition.

In some embodiments, the sensor of the present invention is contacted with a mammal in need of glucose monitoring to detect glucose level. The contacting can include implanting the sensor under skin or mucosa. Alternatively, the contacting can include applying the sensor to a skin or mucosa surface. For instance, the method can include contacting a sensor with a skin or mucosa surface of a mammal in need of analyte monitoring to detect at least one analyte, wherein the sensor comprises a holder containing at least one organism that reacts with the at least one analyte.

In some embodiments, the sensor is used to diagnose diabetes from the glucose level. In certain embodiments, the sensor is used to treat diabetes in response to the glucose level. Diabetes treatments are known in the art. For instance, the diabetes treatment can include administering insulin to a mammal in need thereof in response to the glucose level.

In other embodiments, obesity can be treated in response to the glucose level. In certain embodiments, caloric intake of a mammal is adjusted in response to the glucose level. In some embodiments, the glucose level is measured as part of a health assessment of a mammal.

The sensor of the present invention can also be used as a laboratory tool in the form of a dip probe for the routine measurement of glucose or other chemicals in a variety of test solutions. Because of the relatively low manufacturing cost and compact size of such sensors, such sensors can be configured for a single use and then disposed after each use.

In view of the above, the present invention may provide one or more of the following advantages. In some embodiments, the sensing technology is low cost and optionally disposable. In certain embodiments, the sensor is non-invasive. In other embodiments, the sensor is suitable for implantation. In some preferred embodiments, the sensor provides a fast response, real-time electrical signal representing blood glucose concentration. In certain embodiments, the sensor is low maintenance. In preferred embodiments, the sensor poses minimal risk to the subject. In other preferred embodiments, the present invention provides an integrated, cost-effective, rapid, and unobtrusive assessment of a subject's medical condition.

In some embodiments, risks to the subject are limited to sensor failure and the potential risk of infection from the organism used to construct the sensor if the organism is released from the holder. Careful selection of the organism reduces the hazard of infection. Additionally, organisms can be selected that are most responsive to benign drug treatments in case of infection. Diagnostics can be devised to verify sensor functionality for both non-invasive and implanted configurations.

The present invention will be further illustrated by way of the following Example. This example is non-limiting and does not restrict the scope of the invention.

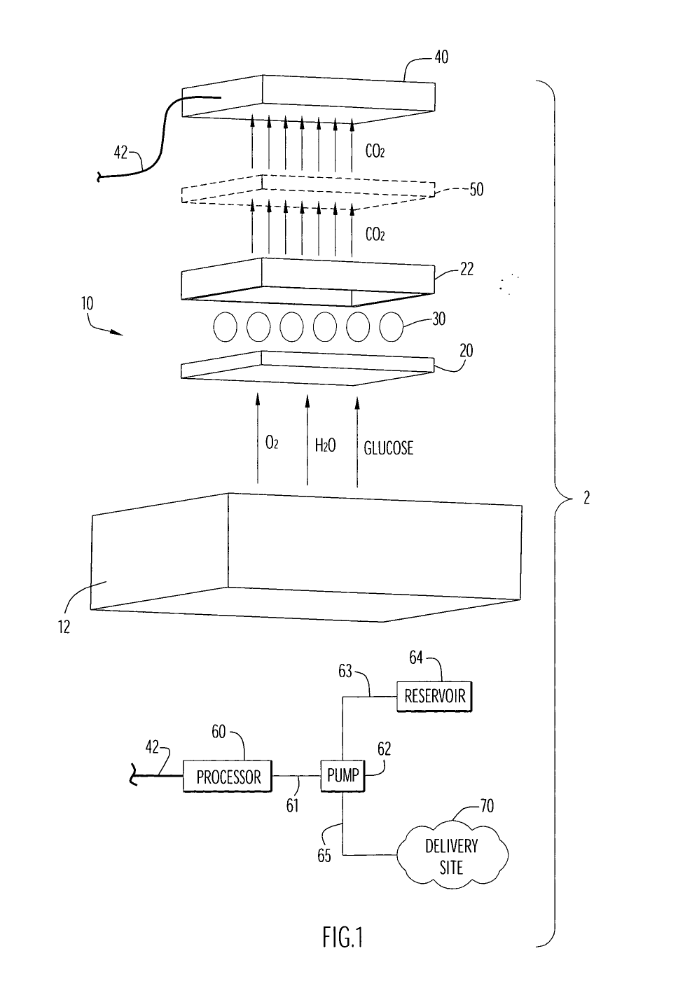

A glucose monitoring system is depicted in FIG. 1. In particular, the system 2 includes a blood glucose sensor 10 configured for use by application to a skin surface 12 of a mammal. Alternatively, as noted above, the sensor can be configured as an implanted device at a suitable location within the body of the mammal. The sensor 10 is constructed by layering and integrating different thin film materials into a composite system. Two layers of semi-permeable membrane 20, 22 are bonded together on their edges to envelope a culture of yeast 30 in a sealed chamber formed between the membranes. The semi-permeable membranes can be constructed of any of the materials described above that permit diffusion of certain chemical compounds while preventing the yeast from escaping the sealed chamber. A thin film detector 40 is secured to the top of the semi-permeable membrane 22. Optionally, a transparent semi-permeable insulator 50 is placed between the semi-permeable membrane 22 and the thin film detector 40 to shield the sealed chamber from the detector while allowing diffusion of certain compounds (e.g., carbon dioxide) through the insulator material.

The sensor 10 operates by using yeast 30 that metabolize oxygen, water, glucose, and potentially other substances. The semi-permeable membranes 20, 22 are configured to facilitate diffusion of these substances, which are necessary to nourish and sustain the yeast, and also the yeast's metabolic waste products, in particular carbon dioxide, through these membranes. The metabolic outputs from the yeast 30 can be quantified using the thin film detector 40 and correlated to measure blood glucose levels. In particular, the diffusion of carbon dioxide generated within the chamber of the sensor, due to metabolic reactions of the yeast 30 as a result of glucose diffusing into the chamber, is controlled so as to pass through membrane 22 for transfer to the detector 40.

The thin film detector 40 is constructed and integrated so as to detect carbon dioxide concentrations. The thin film detector 40 converts the carbon dioxide concentration into an output signal that is carried through at least one signal lead 42. Any suitable carbon dioxide detector can be implemented into the sensor 10, such as any of the types described above.

The detector 40 is connected to a processor 60, via the signal lead(s) 42, to facilitate the transfer of information regarding the amount of carbon dioxide generated, which the processor then utilizes to determine the amount of glucose present in the blood stream at a particular area of the body of the mammal. The processor 60 also communicates with a pump 62, via a communication link 61 (e.g., electrical wire or wireless communication), to control operation of the pump based upon determined glucose levels within the mammal. The pump 62 is connected, via a fluid line 63, to a reservoir 64 that contains one or more drugs (e.g., insulin) that can be delivered by the pump into the mammal. The pump 62 is controlled by the processor 60 to deliver a suitable amount of a drug, via a supply line 65, to a suitable delivery site 70 (e.g., an injection location) within the mammal's body. Thus, the system 2 can selectively adjust and control the delivery of an agent (e.g., insulin) into the mammal's body based upon a measured concentration of glucose within the bloodstream of the mammal. The foregoing embodiments and advantages are merely exemplary and are not to be construed as limiting the present invention. The description of the present invention is intended to be illustrative, and not to limit the scope of the claims. Many alternatives, modifications, and variations will be apparent to those skilled in the art.

Having described preferred embodiments of new and improved composite thin-film glucose sensor, it is believed that other modifications, variations and changes will be suggested to those skilled in the art in view of the teachings set forth herein. It is therefore to be understood that all such variations, modifications and changes are believed to fall within the scope of the present invention as defined by the appended claims and their equivalents. Although specific terms are employed herein, they are used in a generic and descriptive sense only and not for purposes of limitation.

1. A sensor system comprising:
a holder comprising at least one semi-permeable layer that forms a chamber;
at least one reactant that reacts with at least one analyte, the at least one reactant being contained within the chamber; and
a detector disposed proximate the at least one semi-permeable layer and configured to detect and measure a concentration of a reaction product from reaction of the at least one reactant with the at least one analyte;
wherein the at least one semi-permeable layer allows passage of the analyte into the chamber and allows passage of the reaction product to the detector.

2. The system of claim 1, wherein the at least one semi-permeable membrane comprises at least two semi-permeable membranes that are bonded together to form the chamber.

3. The system of claim 1, wherein the at least one semi-permeable membrane is permeable to oxygen, water, carbon dioxide and glucose.

4. The system of claim 3, wherein the at least one reactant comprises yeast.

5. The system of claim 5, wherein the reaction product detected by the detector includes carbon dioxide.

6. The system of claim 5, wherein the detector comprises an aqueous layer, and a carbon dioxide concentration within the aqueous layer is determined by measuring pH within the aqueous layer.

7. The system of claim 1, further comprising a transparent insulator disposed between the holder and the detector.

8. The system of claim 1, further comprising a skin or mucosa permeation enhancer adjacent to the holder.

9. The system of claim 8, wherein the permeation enhancer comprises an iontophoresis generator.

10. The system of claim 8, wherein the skin or mucosa permeation enhancer comprises a composition that increases skin or mucosa permeability.

11. The system of claim 1, further comprising an adhesive layer configured to affix the holder to a skin or mucosa surface of a mammal.

12. The system of claim 1, further comprising:
a pump;
a reservoir including an agent and connected with the pump; and
a controller in communication with the detector and the pump;
wherein the controller controls the pump to facilitate delivery of a selected amount of agent to a delivery site, via the pump, based upon the measured concentration of the reaction product.

13. The system of claim 12, wherein the agent comprises at least one of insulin, glucose and glucagon.

14. A method of monitoring an analyte within a mammal, comprising:
contacting the holder of the system of claim 1 with a mammal to facilitate diffusion of the analyte from the mammal through the at least one semi-permeable membrane for reaction with the reactant within the chamber of the holder; and
measuring a concentration of reaction product that diffuses from the chamber to the detector.

15. The method of claim 14, wherein the contacting comprises applying the holder to a skin or mucosa surface of the mammal.

16. The method of claim 14, wherein the contacting comprises implanting the holder under skin or mucosa of the mammal.

17. The method of claim 14, wherein the analyte comprises glucose, the reaction product comprises carbon dioxide, and the method further comprises:
delivering at least one of insulin, glucose and glucagon into the blood stream of the mammal based upon the measured concentration of carbon dioxide by the detector.
